(19) United States
(12) Patent Application Publication
(10) Pub. No.: US 2025/0177727 A1
(43) Pub. Date: Jun. 5, 2025

(54) SYSTEM FOR TRACKING REAL-TIME POSITIONS OF A MOVABLE SENSOR WITHIN A PATIENT'S BODY

(71) Applicant: ABIOMED Europe GmbH, Aachen (DE)

(72) Inventors: Katrin Lunze, Aachen (DE); Paul Zech, Aachen (DE)

(21) Appl. No.: 18/846,060

(22) PCT Filed: Mar. 15, 2023
(86) PCT No.: PCT/EP2023/056540

§ 371 Date: Sep. 11, 2024

(30) Foreign Application Priority Data
Mar. 15, 2022 (EP) 22162195.6

(51) Int. Cl. A61M 60/867; A61B 5/06; A61M 25/01; A61M 25/09; A61M 60/13

(52) U.S. Cl. CPC A61M 60/867; A61B 5/062; A61M 25/0127; A61M 60/13; A61M 25/09

(57) ABSTRACT

The present invention relates to a tracking system (10) comprising: an emitting device (12) configured to establish a measurement volume within at least a part of a patients body, the patients body having a median plane, a transverse plane, and a coronal plane; a movable sensor (14) which is movable within the measurement volume; a first reference sensor (16) establishing a global coordinate system within the measurement volume, the global coordinate system having an origin, an x-axis, a y-axis and a z-axis; and a controller (18). The controller (18) is configured to detect real-time positions of the movable sensor (14) within the global coordinate system, to set the origin of the global coordinate system as an anchor point in relation to the patients body, to span a first reference plane in direction of the x-axis and the z-axis being parallel to the median plane, to span a second reference plane in direction of the y-axis and the z-axis being parallel to the transverse plane, to span a third reference plane in direction of the x-axis and the y-axis being parallel to the coronal plane, and preferably to translate in parallel at least one of the first reference plane, the second reference plane and the third reference plane based on an expected movement path of the movable sensor.

### BACKGROUND

The present invention relates to a tracking system comprising an emitting device, a movable senor, a first reference sensor and a controller. The present invention further relates to a method of tracking real-time positions of a movable sensor within a patient's body with an according tracking system. In particular, the tracking system is to be used when introducing a blood pump or a guidewire for a blood pump into a patient's body. The blood pump may be a catheter pump, in particular an intravascular blood pump, an intracardiac blood pump or any other kind of ventricular assist device.

Such blood pumps of different types are known from the prior art and are intended to support the function of a patient's heart, either in a short-term application, in which an intravascular blood pump is placed in the patient for a couple of days or weeks, or in a long-term application, in which the intravascular blood pump is placed in the patient for a couple of weeks or months. The blood pumps may e.g. be inserted into a patient's body through the aorta by using a catheter, or may be placed in the thoracic cavity. During planned surgery, intravascular blood pumps are often introduced into the patient's body using a guidewire, serving as a track for the intravascular blood pump. For example, guidewires and intravascular blood pumps are introduced into the patient's body using fluoroscopy to warrant a correct positioning of the intravascular blood pump in the patient's left ventricle. Commonly, X-ray is used for fluoroscopy.

However, there are certain disadvantages associated with fluoroscopy and in particular with X-ray. During planned surgery for placing the blood pump or the guidewire respectively X-ray is not available without restrictions. In addition, the patient and the physician are exposed to a high amount of radiation when conducting X-ray.

Therefore, the need exists to provide an ameliorated solution for tracking a position of a blood pump or a guidewire for a blood pump when introducing the blood pump or guidewire into a patient's body.

### SUMMARY OF THE INVENTION

According to a first aspect, a tracking system comprises an emitting device configured to establish a measurement volume within at least a part of a patient's body, a movable sensor which is movable within the measurement volume and a first reference sensor establishing a global coordinate system within the measurement volume. The global coordinate system comprises an origin, an x′-axis, a y′-axis and a z′-axis. The patient's body has a median plane, a transverse plane and a coronal plane.

The tracking system according to the first aspect further comprises a controller and the controller is configured to detect real-time positions of the movable sensor within the global coordinate system, to set the origin of the global coordinate system as an anchor point in relation to the patient's body, to span a first reference plane in direction of the x′-axis and the z′-axis being parallel to the median plane, to span a second reference plane in direction of the y′-axis and the z′-axis being parallel to the transverse plane, to span a third reference plane in direction of the x′-axis and the y′-axis being parallel to the coronal plane, and to translate in parallel at least one of the first reference plane, the second reference plane and the third reference plane based on an expected movement path of the movable sensor. The first reference sensor may be attached to the patient's skin and may be positioned on the upper part of the sternum on the manubrium.

Here, the median plane, the transverse plane and the coronal plane of the patient's body are the three main planes of a human body and the controller can set up the reference planes being parallel to the main planes, as the first reference sensor is the anchor point for the tracking procedure. The expected movement path of the movable sensor is known to the physician, as the movable sensor is introduced into the patient's body e.g. via a right axillary access, a left axillary access or a right or left femoral access. Thus, at least one of the reference planes may be moved in parallel to delimit a volume within the measurement volume the movable sensor shall not exit during insertion of e.g. the intravascular blood pump or the guidewire the movable sensor is attached to. Further, as the emitting device generates the measurement volume and as the positions of the movable sensor and the first reference sensor within the measurement volume can be detected, it can also be tracked if the movable sensor is within the delimited volume obtained by moving at least one of the reference planes. This allows the physician a better orientation and support when inserting the guidewire or intravascular blood pump respectively.

In order to set the origin of the global coordinate system as an anchor point in relation to the patient's body, it is possible to define the origin of the global coordinate system as a point of intersection of the median plane, the transverse plane, and the coronal plane.

Preferably, the first reference plane, the second reference plane and the third reference plane establish eight octants within the measurement volume. Preferably, the controller is configured to adjust the partition of the octants based on the expected movement path of the movable sensor. In moving one or more of the reference planes, the size of each of the octants can be changed. For example, one of the octants may be of particular interest to the physician as covering the expected movement path of the movable sensor and hence, the space delimited. This may allow for a better orientation, as “leaving” the octant of interest with the movable sensor can directly be recognized by the physician. For example, in case a guidewire having the movable sensor is introduced into the patient's body via a left axillary access, the octant covering the left axillary access is sized or enlarged respectively by moving the respective reference planes so that it covers the entire vascular system of interest and the left ventricle.

Preferably, the tracking system comprises a second reference sensor and the controller is configured to detect a position and/or an orientation of the second reference sensor within the global coordinate system. The second reference sensor may be used as a further aid for the physician, as its position is known to the physician. For instance, the second reference sensor may be positioned on the patient's skin above the apex of the heart.

The controller may further be configured to align the x′-axis in parallel with a line of intersection of the coronal plane and the median plane, and/or to align the y′-axis in parallel with a line of intersection of the transverse plane and the coronal plane, and/or to align the z′-axis in parallel with a line of intersection of the median plane and the transverse plane.

The first reference sensor may further be configured to be manually aligned with the median plane and/or the transverse plane and/or the coronal plane such that the x′-axis is parallel with a line of intersection of the coronal plane and the median plane, and/or such that the y′-axis is parallel with a line of intersection of the transverse plane and the coronal plane, and/or such that z′-axis is parallel with a line of intersection of the median plane and the transverse plane. For example, the first reference sensor may comprise markings or indicia indicating the x′-axis and/or the y′-axis, such as crossing lines. The markings or indicia may be visible on an outer surface of a housing of the first reference sensor. Further, the first reference sensor may comprise a water level in order to align the z′-axis. This water level may, for example, be attached to a housing of the first reference sensor.

The tracking system may further comprise a third reference sensor and the controller may be configured to set an orientation of the median plane, an orientation of the transverse plane, and/or an orientation of the coronal plane within the measurement volume based on the orientation of the third reference sensor. Generally, the patient is lying on a surgical table or the like so that the coronal plane is parallel to the surface of the surgical table and the median plane and the transverse plane are perpendicular to the surgical table. In case the emitting device is disposed in or on the surgical table, the three main planes of the patient's body can easily be set. However, in case a mobile emitting device is used, the position of the emitting device relative to the patient's body may not be known exactly. By using a third reference sensor fixed e.g. to the surface of the surgical table, the three main planes can easily be set by the controller as the orientation of the main planes within the measurement volume is thus known. Preferably, the third reference sensor is at least a three degrees of freedom sensor denoting the x-direction, the y-direction and the z-direction.

Preferably, the controller is configured to detect a crossing of the movable sensor with at least one of the first reference plane, the second reference plane and the third reference plane. The controller may be configured to emit a warning signal to the physician in case a crossing is detected. This allows the physician to directly notice a “leaving” of the movable sensor of the expected movement path of the movable sensor.

The controller may be configured to establish a target volume within the global coordinate system. The target volume may be a suitable volume denoting the final position of the movable sensor along the expected movement path, e.g. the left ventricle of the patient's heart. The controller may virtually generate a target volume denoting the volume or region of interest, i.e. the final position of the movement sensor. The target volume may have any suitable shape, e.g. a cylindric shape, a ball shape, a cone shape, a football shape or an egg shape. This may further help the physician in placing e.g. the guidewire with the thereto attached movable sensor on its final position.

The controller may further be configured to establish a spatially limited auxiliary plane, the auxiliary plane may indicate a unique structure within the patient's body. For instance, the unique structure may be the aortic valve and the auxiliary plane may virtually be generated by the controller so that the aortic valve largely corresponds in its position to the position of the auxiliary plane. This may further help the physician in placing e.g. the guidewire with the thereto attached movable sensor on its final position.

The first reference sensor may be a six degrees of freedom sensor. The second reference sensor may be a six degrees of freedom sensor. The third reference sensor may be a six degrees of freedom sensor. The movable sensor, the first reference sensor, the second reference sensor and/or the third reference sensor may be an embedded electromagnetic sensor. The movable sensor may be a five degrees of freedom sensor or a six degrees of freedom sensor. This allows for an overall high resolution.

Preferably, the tracking system further comprises a medical device, wherein the movable sensor is attached to the medial device. The medical device may be an intravascular blood pump, an intracardiac blood pump, a catheter blood pump or a guidewire, for example for an intravascular blood pump.

The emitting device may be an electromagnetic field generator. The electromagnetic field generator is preferably configured to emit a low-intensity varying electromagnetic field that establishes the measurement volume. Said electromagnetic field induces small currents within the sensors, i.e. within the movable sensor, within the first reference sensor, within the second reference sensor and/or within the third reference sensor. The induced currents are relayed to the controller and the controller is preferably configured to amplify and digitalize the currents for further processing as a digital signal.

The tracking system may further comprise a display device. The display device may be a suitable device, e.g. a TFT-device or an LCD-device. The display device may be configured to display the real-time positions of the movable sensor and/or to display the first reference plane and/or to display the second reference plane and/or to display the third reference plane and/or to display the target volume and/or to display the auxiliary plane in the global coordinate system and/or to display the octants. The display device may, in particular, be configured to display the real-time positions of the movable sensor in relation to the first reference plane and/or in relation to the second reference plane and/or in relation to the third reference plane and/or in relation to the target volume and/or in relation to the auxiliary plane in the global coordinate system and/or in relation to the octants. Thus, a real-time position of the movable sensor in relation to the further entities may be visualized to the physician via the display device. This may further allow the physician a better orientation when inserting e.g. the guidewire with the thereto attached movable sensor into the patient's body.

The tracking system may further comprise an input device. The input device may be configured to communicate with the controller. The input device may be a suitable device, e.g. a remote control, a smartphone, a keyboard, a terminal, a personal computer, a tablet or the like. Further, the display device may comprise the input device, e.g. as a touch-screen.

According to a second aspect, a method of tracking real-time positions of a movable sensor with a tracking system, for example with a tracking system according to the first aspect, comprises the following steps: providing an emitting device configured to establish a measurement volume within at least a part of a patient's body, the patient's body having a median plane, a transverse plane, and a coronal plane; providing a movable sensor which is movable within the measurement volume; providing a first reference sensor establishing a global coordinate system within the measurement volume, the global coordinate system having an x′-axis, a y′-axis and a z′-axis; providing a controller; attaching the reference sensor on the skin of the patient's body; spanning, with the controller, a first reference plane in direction of the x′-axis and the z′-axis being parallel to the median plane; spanning, with the controller, a second reference plane in direction of the y′-axis and the z′-axis being parallel to the transverse plane; spanning, with the controller, a third reference plane in direction of the x′-axis and the y′-axis being parallel to the median plane; translating, with the controller, in parallel at least one of the first reference plane, the second reference plane and the third reference plane based on an expected movement path of the movable sensor; detecting, with the controller, real-time positions of the movable sensor within the global coordinate system. The first reference sensor may be positioned on the upper part of the sternum on the manubrium.

In order to set the origin of the global coordinate system as an anchor point in relation to the patient's body, the origin of the global coordinate system may be defined as a point of intersection of the median plane, the transverse plane, and the coronal plane.

Here, the median plane, the transverse plane and the coronal plane of the patient's body are the three main planes of a human body and the reference planes are set up being parallel to the main planes, as the first reference sensor is the anchor point for the tracking procedure. The expected movement path of the movable sensor is known to the physician, as the movable sensor is introduced into the patient's body e.g. via a right axillary access, a left axillary access or a right or left femoral access. Thus, at least one reference plane is moved in parallel to delimit a volume within the measurement volume the movable sensor shall not exit during introduction of e.g. the intravascular blood pump or guidewire the movable sensor may be attached to. Further, as the emitting device generates the measurement volume and the positions of the movable sensor and the first reference sensor within the measurement volume can be detected, it can also be tracked if the movable sensor is within the delimited area obtained by moving at least one of the reference planes. This allows the physician a better orientation and aid when inserting the guidewire or intravascular blood pump respectively.

The method may further comprise the steps of establishing, with the controller, eight octants within the measurement volume based on the first reference plane, the second reference plane and the third reference plane, and adjusting, with the controller, the partition of the octants based on the expected movement path of the movable sensor. One of the octants may be of particular interest to the physician as covering the expected movement path of the movable sensor and hence, the space delimited. This may allow for a better orientation, as “leaving” the octant of interest with the movable sensor can directly be recognized by the physician. For example, in case a guidewire having the movable sensor is introduced into the patient's body via a left axillary access, the octant covering the left axillary access is sized or enlarged respectively by moving the respective reference planes so that it covers the entire vascular system of interest and the left ventricle of the heart.

The method may further comprise the steps of providing a second reference sensor, and detecting, with the controller, the position of the second reference sensor within the global coordinate system. The second reference sensor is used as a further aid for the physician, as its position is known to the physician. For instance, the second reference sensor may be positioned on the patient's skin above the apex of the heart.

The method may further comprise aligning the x′-axis in parallel with a line of intersection of the coronal plane and the median plane, and/or aligning the y′-axis in parallel with a line of intersection of the transverse plane and the coronal plane, and/or aligning the z′-axis in parallel with a line of intersection of the median plane and the transverse plane.

The method may further comprise manually aligning the first reference sensor with the median plane and/or the transverse plane and/or the coronal plane such that the x′-axis is parallel with a line of intersection of the coronal plane and the median plane, and/or such that the y′-axis is parallel with a line of intersection of the transverse plane and the coronal plane, and/or such that z′-axis is parallel with a line of intersection of the median plane and the transverse plane.

The method may further comprise the steps of providing a third reference sensor, and setting, with the controller, an orientation of the median plane, an orientation of the transverse plane, and/or an orientation of the coronal plane within the measurement volume based on the orientation of the third reference sensor. Generally, the patient is lying on a surgical table or the like so that the coronal plane is parallel to the surface of the surgical table and the median plane and the transverse plane are perpendicular to the surgical table. In case the emitting device is disposed in or on the surgical table, the three main planes of the patient's body can easily be set. However, in case a mobile emitting device is used, the position of the emitting device relative to the patient's body may not be known exactly. By using a third reference sensor fixed e.g. to the surface of the surgical table, the three main planes can easily be set as the orientation of the main planes within the measurement volume is thus known. Preferably, the third reference sensor is at least a three degrees of freedom sensor denoting the x-direction, the y-direction and the z-direction.

The method may further comprise detecting, with the controller, a crossing of the movable sensor with at least one of the first reference plane, the second reference plane and the third reference plane.

The method may further comprise the step of establishing, with the controller, a target volume within the global coordinate system. The target volume may be a suitable volume denoting the final position of the movable sensor along the expected movement path, e.g. the left ventricle of the patient's heart. The target volume may by virtually generated with the controller for depicting the volume or region of interest, i.e. the final position of the movement sensor. The target volume may have any suitable shape, e.g. a cylindric shape, a ball shape, a cone shape, a football shape or an egg shape. This may further help the physician in introducing e.g. the guidewire with the thereto attached movable sensor into its final position.

The method may further comprise the step of establishing, with the controller, a spatially limited auxiliary plane, the auxiliary plane indicating a unique structure within the patient's body. For instance, the unique structure may be the aortic valve and the auxiliary plane may virtually be generated so that the aortic valve largely corresponds in its position to the position of the auxiliary plane. This may further help the physician in introducing e.g. the guidewire with the thereto attached movable sensor into its final position.

The method may further comprise the step of displaying, on a display device, the real-time positions of the movable sensor and/or displaying, on a display device, the first reference plane and/or displaying, on a display device, the second reference plane and/or displaying, on a display device, the third reference plane and/or, displaying, on a display device, the target volume and/or displaying, on a display device the auxiliary plane in the global coordinate system and/or displaying, on a display device the octants. In particular, the real-time positions of the movable sensor in relation to the first reference plane and/or in relation to the second reference plane and/or in relation to the third reference plane and/or in relation to the target volume and/or in relation to the auxiliary plane in the global coordinate system and/or in relation to the octants may be displayed. Thus, real-time position of the movable sensor in relation to the further entities may be visualized to the physician. This further allows the physician a better orientation when inserting e.g. the guidewire with the thereto attached movable sensor into the patient's body.

The moveable sensor may be introduced into the patient's body via the femoral artery or via the axillary artery.

### BRIEF DESCRIPTION OF THE DRAWINGS

The foregoing summary, as well as the following detailed description of exemplary embodiments, will be better understood when read in conjunction with the appended drawings. For the purpose of illustrating the present disclosure, reference is made to the drawings. The scope of the disclosure is not limited, however, to the specific embodiments disclosed in the drawings.

In the drawings:

FIG. 1 is a functional block diagram of an exemplary tracking system, in accordance with aspects of the disclosure,

FIG. 2 depicts a schematic view of a patient's body with a placed reference sensor,

FIG. 3 depicts a schematic view of an alignment of a global coordinate system to three main planes of a human body,

FIG. 4 depicts a schematic view of a virtual model of the patient's body with placed reference planes,

FIG. 5 the view of FIG. 4 with additionally placed target volume,

FIG. 6 the view of FIG. 5 as a top view along the z-axis,

FIG. 7 the view of FIG. 4 with additionally placed auxiliary plane and tracked positions for right axillary access,

FIG. 8 the view of FIG. 7 as a top view along the z-axis,

FIG. 9 the view of FIG. 7 without a virtual model of the patient's body,

FIG. 10 the view of FIG. 9 as a top view along the z-axis,

FIG. 11 depicts a view similar to FIG. 9 with tracked positions for right axillary access, left axillary access and femoral access, and

FIG. 12 the view of FIG. 11 as a top view along the z-axis.

### DETAILED DESCRIPTION

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

To provide an overall understanding of the systems, methods, and devices described herein, certain illustrative examples will be described. Although various examples may describe intravascular blood pumps, it will be understood that the improvements of the present technology may also be adapted and applied to other types of medical devices such as electrophysiology study and catheter ablation devices, angioplasty and stenting devices, angiographic catheters, peripherally inserted central catheters, central venous catheters, midline catheters, peripheral catheters, inferior vena cava filters, abdominal aortic aneurysm therapy devices, thrombectomy devices, TAVR delivery systems, cardiac therapy and cardiac assist devices, including balloon pumps, cardiac assist devices implanted using a surgical incision, and any other venous or arterial based introduced catheters and devices. As is known, intravascular blood pumps can be introduced into a patient, either surgically or percutaneously, to deliver blood from one location in the heart or circulatory system to another location in the heart or circulatory system. For example, when deployed in the left ventricle, an intravascular blood pump can pump blood from the left ventricle of the heart into the aorta. When deployed in the right ventricle, an intravascular blood pump can pump blood from the inferior vena cava into the pulmonary artery.

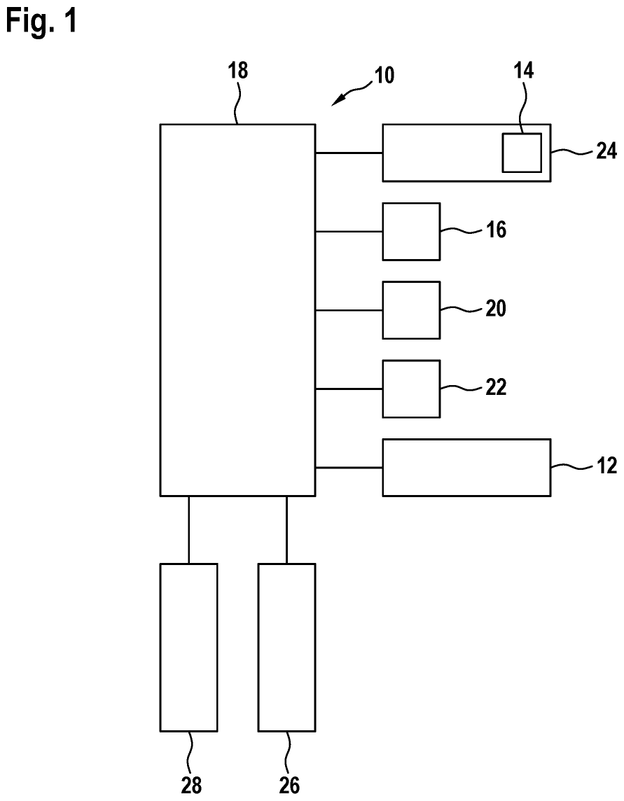

FIG. 1 shows a schematic block diagram of a tracking system 10 according to the invention. The tracking system 10 comprises an emitting device 12, a movable sensor 14, a first reference sensor 16, a second reference sensor 20, a third reference sensor 22, a controller 18, a medical device 24, a display device 26 and an input device 28. Preferably the movable sensor 14 is attached to the medical device 24. The medical device 24 may be an intravascular blood pump or a guidewire for a blood pump. In the following, the medical device 24 will be referred to as a guidewire for an intravascular blood pump.

The first reference sensor 16 may comprise markings or indicia which will be described in more detail below. The markings or indicia may be visible on an outer surface of a housing of the first reference sensor 16. Further, the first reference sensor 16 may comprise a water level. This water level may, for example, be attached to the housing of the first reference sensor 16.

In this embodiment, the emitting device 12 is an electromagnetic field generator configured to establish a measurement volume in that it emits a low-intensity, varying electromagnetic field which induces small currents within the movable sensor 14, the first reference sensor 16, the second reference sensor 20 and the third reference sensor 22 when they are located within the measurement volume. The emitting device 12, the movable sensor 14, the first reference sensor 16, the second reference sensor 20 and the third reference sensor 22 are all connected to controller 18 by suitable means, e.g. by a cable or wirelessly. The controller 18 amplifies and digitalizes the received currents and calculates the position of the movable sensor 14, of the first reference sensor 16, of the second reference sensor 20 and of the third reference sensor 22 as will be explained in more detail below.

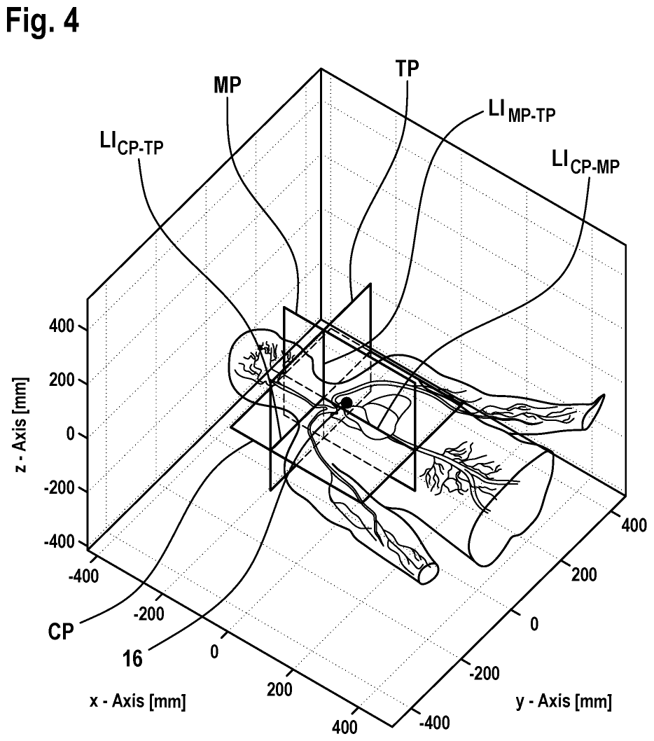

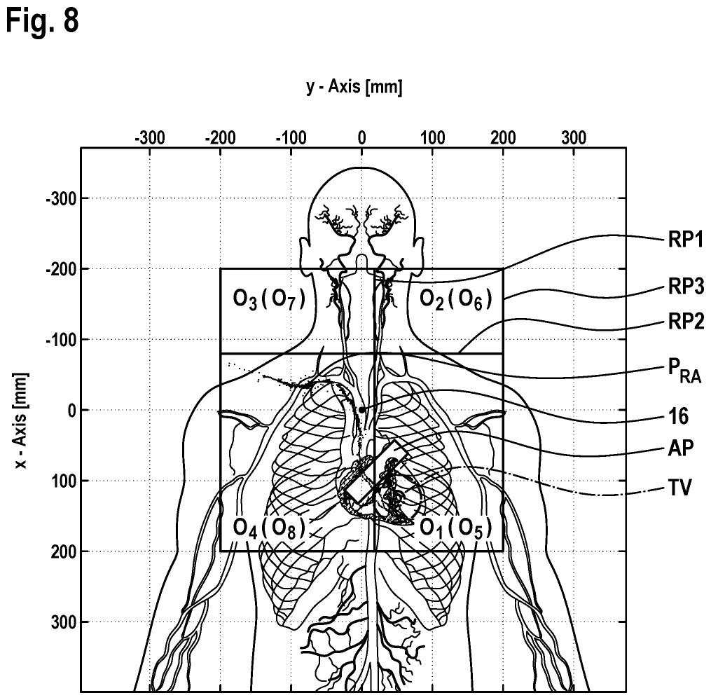

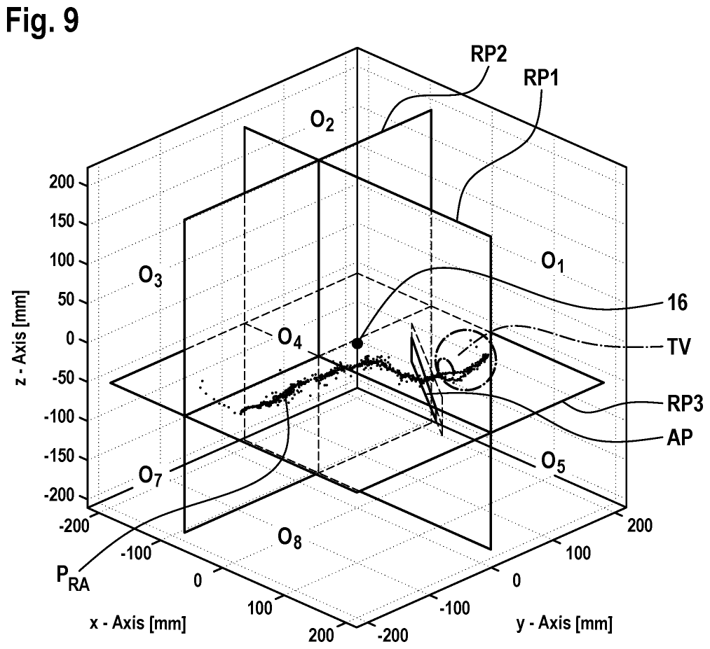

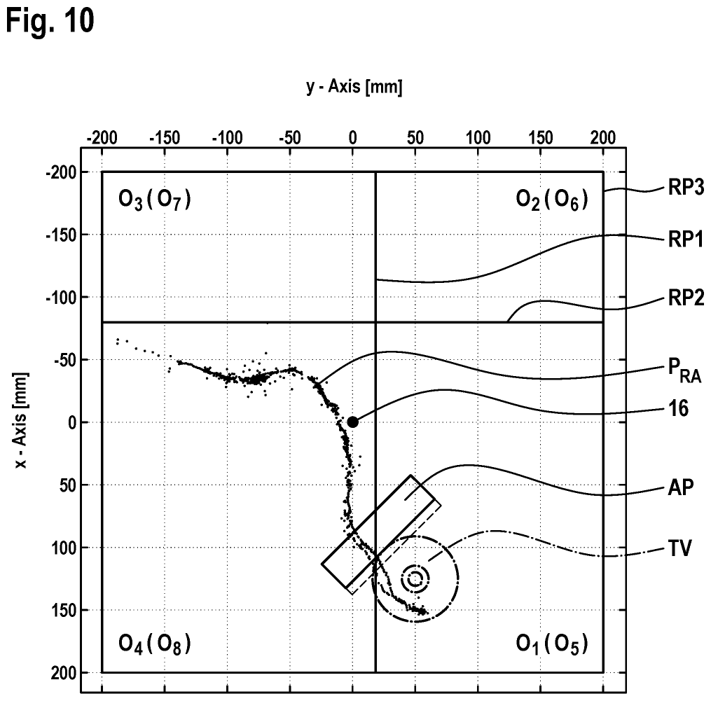

The display device 26 is also connected to the controller 18 and the controller may be configured to display various information on the display device 26. The display device 26 may be a TFT-display. To operate the tracking system 10, an input device 28 is provided. The input device 28 may be a keyboard, a terminal, a smartphone, a tablet or the like, which is connected to the controller 18 in a suitable way. The display device 26 may comprise the input device 28 and may thus be a touchscreen. The illustrations shown in FIGS. 4 to 12 also depict examples of possible visualizations on the display device 26.

Prior to introducing the guidewire 24 into a patient's body B, the patient is placed lying on an operating table or the like, with the patient's coronal plane CP being parallel to the surface of the operating table and with the patient's median plane MP being perpendicular to the surface of the operating table. The patient's transverse plane TP is perpendicular to the patient's coronal plane CP and to the patient's median plane MP. FIG. 4 depicts a view with a virtual model of the patient's body showing the patient's coronal plane CP, the patient's median plane MP and the patient's transverse plane TP.

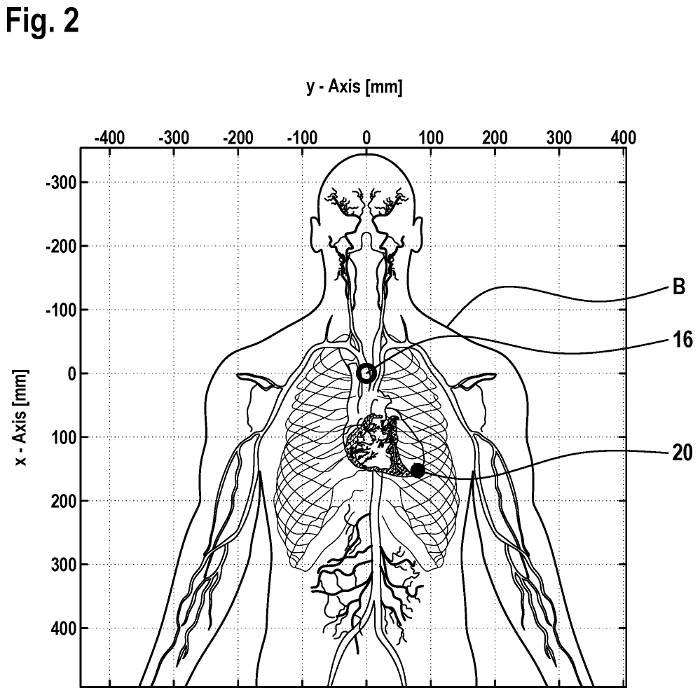

The first reference sensor 16 may then be placed on the upper part of the sternum on the manubrium of the patient's body B, see FIG. 2. The first reference sensor 16 may be a five degrees of freedom sensor or a six degrees of freedom sensor. To improve the accuracy and to adapt to the patient's individual anatomy, the second reference sensor 20 may be positioned on the patient's body B above the apex of the heart, see FIG. 2. The second reference sensor 20 may be a five degrees of freedom sensor or a six degrees of freedom sensor. To ensure the correct position of the second reference sensor 20, the apex of the heart may be determined prior to surgery e.g. by a transthoracic echocardiogram or ultrasound imaging.

The electromagnetic field generator 12 may be placed in vicinity of the patient's body, so that the part of interest of the patient's body B is located within the measurement volume established by the electromagnetic field generator 12. In particular, there are two possible solutions for the electromagnetic field generator 12, namely a field generator 12 fixed in space in a known manner or a field generator fixed in space in an unknown manner. The electromagnetic field generator 12 may be fixed to the operating table so that its position in space, in particular its position relative to the operating table, is known. Further, the electromagnetic field generator 12 may also be a mobile field generator, e.g. movably attached to an arm or the like, so that its position in space, in particular its position relative to the operating table, is not exactly known.

In case a mobile electromagnetic field generator 12 is used, the third reference sensor 22 may be necessary to determine the surface of the operating table the patient's body is lying on. Therefore, the third reference sensor 22 may be placed on the surface of the operating table. As the third reference sensor 22 is used only as an auxiliary resource, it is sufficient that the third reference sensor 22 is a three degrees of freedom sensor. Of course, it is also possible to use a five degrees of freedom sensor or a six degrees of freedom sensor as the third reference sensor 22. By means of the third refence sensor 22, the coordinate system underlying the measurement volume is aligned by the controller 18 so that the x-y-plane is parallel to the surface of the operating table. The coordinate system of the measurement volume has an x-axis, a y-axis and a z-axis.

The position of the first reference sensor 16 is now the anchor point and establishes a global coordinate system COS having an x′-axis, a y′-axis and a z′-axis. Further, the first reference sensor 16 sets the anatomy of the patient's body B in reference to the global coordinate system COS. All other positions, e.g. of the movable sensor 14, are shown and calculated by the controller 18 in reference to the global coordinate system COS.

An orientation of the patient's coronal plane CP, an orientation of the patient's transverse plane TP and an orientation of the patient's median plane MP may be known to the physician because the patient is typically lying on an operating table so that the orientation of the coronal plane CP, of the transverse plane TP and of the median plane MP corresponds to an orientation of the operating table.

For example, an origin of the global coordinate system (COS) is defined as a point of intersection of the median plane MP, the transverse plane TP, and the coronal plane CP.

In a first step, the global coordinate system COS of the first reference sensor 16 is adapted to the operating table and hence, to the coordinate system underlying the measurement volume established by the electromagnetic field generator 12.

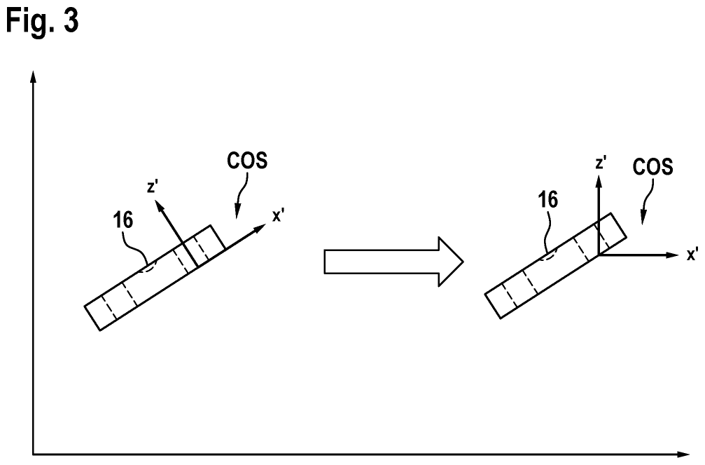

Preferably, this adaption is done automatically by means of the controller 18. After placement of the first reference sensor 16 on the manubrium of the patient's body B, the global coordinate system COS is established so that e.g. the x′-axis may be inclined relative to the patient's coronal plane CP due to the slope of the thorax of the patient's body B. Hence, the x′-axis may also be inclined to the surface of the operating table being parallel to the patient's coronal plane CP, see left part of FIG. 3. Accordingly, the global coordinate system COS of the first reference sensor 16 is recalibrated by the controller 18 so that the x′-axis is parallel to a line of intersection (LI_{CP-MP}) of the patient's coronal plane CP and the median plane MP, the y′-axis is parallel to a line of intersection (LI_{CP-TP}) of the patient's transverse plane TP and the coronal plane CP, and the z′-axis is parallel to a line of intersection (LI_{MP-TP}) of the patient's median plane MP and the transverse plane TP, see right part of FIG. 3. In other words, the global coordinate system COS is now fully in parallel (i.e. aligned with) with the coordinate system underlying the measurement volume established by the electromagnetic field generator 12 and thus, with the operating table. Hence, the x′-axis of the global coordinate system COS coincides with the x-axis of the coordinate system of the measurement volume, the y′-axis of the global coordinate system COS coincides with the y-axis of the coordinate system of the measurement volume and the z′-axis of the global coordinate system COS coincides with the z-axis of the coordinate system of the measurement volume.

In addition or as an alternative to the automatic adaption of the global coordinate system COS to the operating table, an orientation of the first reference sensor 16 may be manually aligned to an orientation of the operating table using the markings or indicia on the housing of the first reference sensor 16 and further using the water level attached to the housing of the first reference sensor 16.

This way, the first reference sensor 16 may be manually aligned with the patient's median plane MP, the patient's transverse plane TP and the patient's coronal plane CP such that the x′-axis is parallel to a line of intersection (LI_{CP-MP}) of the patient's coronal plane CP and the median plane MP, and such that the y′-axis is parallel to a line of intersection (LI_{CP-TP}) of the patient's transverse plane TP and the coronal plane CP, and such that the z′-axis is parallel to a line of intersection (LI_{MP-TP}) of the patient's median plane MP and the transverse plane TP.

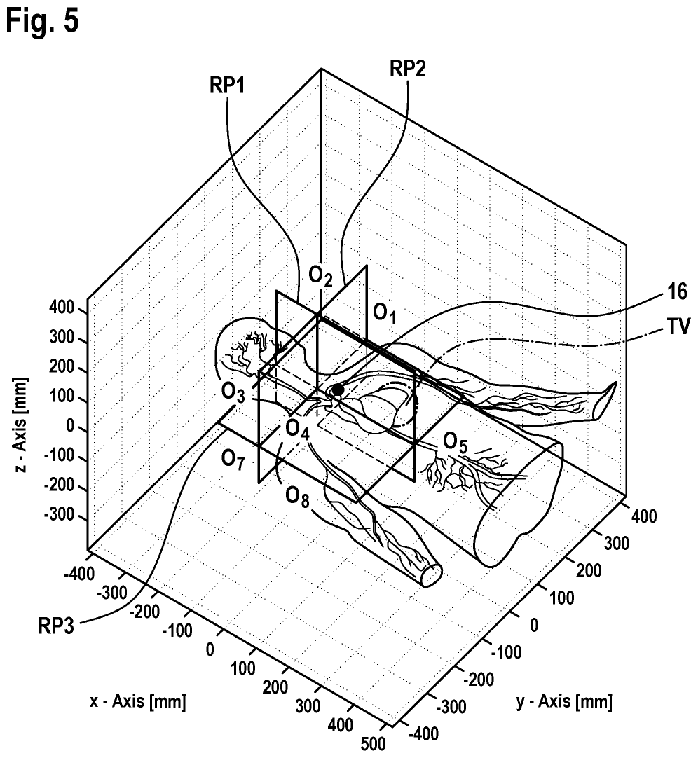

Next, the controller 18 spans three reference planes RP1, RP2 and RP3 within the global coordinate system COS. A first reference plane RP1 is spanned in direction of the x-axis and the z-axis in parallel to the patient's median plane MP, a second reference plane RP2 is spanned in direction of the y-axis and the z-axis parallel to the patient's transverse plane TP and a third reference plane RP3 is spanned in direction of the x-axis and the y-axis in parallel to the patient's coronal plane CP, see FIG. 5. The three reference planes RP1, RP2, and RP3 are all perpendicular to each other and in total delimit eight areas in form of octants O₁ to O₈ within the measurement volume. The first reference plane RP1 may be identical to the patient's median plane MP, the second reference plane RP2 may be identical to the patient's transverse plane TP and the third reference plane RP3 may be identical to the patient's coronal plane CP.

Of course, an expected movement path of the movable sensor 14 within the patient's body B is known to the physician prior to surgery, as the physician is well aware of the placement method of the guidewire 24 carrying the movable sensor 14. For describing the next steps, it is assumed that a right axillary access is used for introducing the guidewire 24 into the patient's body.

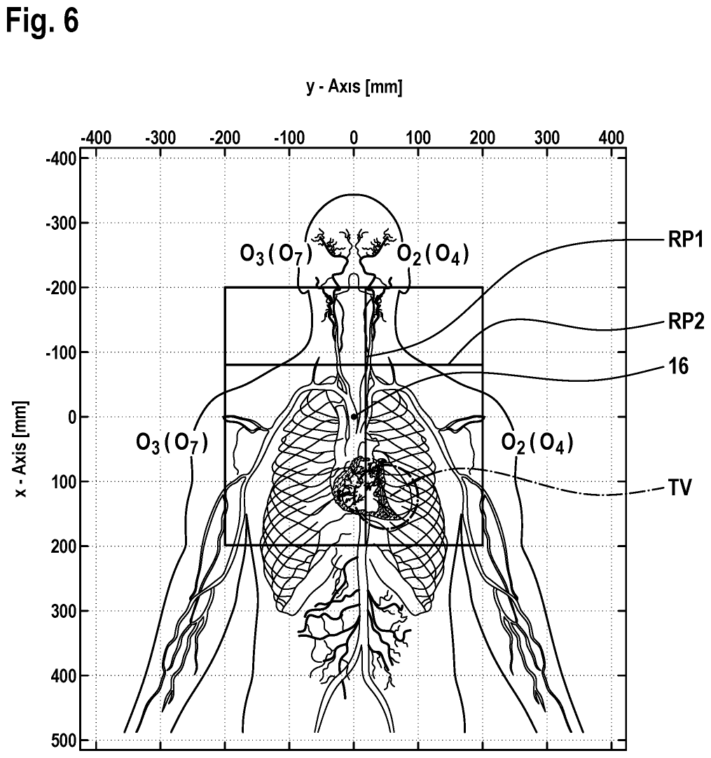

As shown in FIGS. 5 and 6, the controller 18 translates in parallel at least one of the first reference plane RP1, the second reference plane RP2 and the third reference plane RP3 on the expected movement path of the guidewire 24 and hence, of the movable sensor 14, given in the global coordinate system COS. In doing so, the partition of the octants O₁ to O₈ relative to each other is adjusted. In the example for a right axillary access the octant O₁ in the front top of FIG. 5 delimits the part of interest of the patient's body within the measurement volume. Accordingly, the first reference plane RP1 is translated a specific amount (e.g. a few millimeters) in direction of the positive y-axis to separate the right axillary from the left axillary and the ascending aorta from the descending aorta. Further, the second reference plane RP2 is translated a specific amount (e.g. a few centimeters) in direction of the negative x-axis to limit the insertion space in direction of the blood vessels leading to the patient's head. Further, the third reference plane RP3 is translated a specific amount (e.g. a few centimeters) in direction of the negative z-axis to separate the ascending aorta and the descending aorta. Hence, the octant O₁ of interest is enlarged and delimits the space in which the expected movement path of the movable sensor 14 and hence the guidewire 24 is shown.

The controller 18 is further configured to detect any crossing of the movable sensor 14 and the reference planes RP1, RP2 and RP3. In other words, the controller 18 can detect if the movable sensor 14 leaves the octant of interest and may emit a warning signal to the physician.

To further aid the physician during the insertion procedure of the guidewire 24 into the patient's body B, the controller 18 may be configured to establish a target volume TV and to visualize the same on the display device 26, see FIGS. 5 to 12. The target volume TV represents the most-likely final position of the guidewire 24, e.g. the left ventricle, after the guidewire 24 has been inserted. The target volume may have any suitable shape, e.g. a ball-shape, a cone-shape, a cylindrical shape or a football-shape. In principle, the target volume TV may be established taking into account a position of the second reference sensor 20. It is also conceivable to set the target volume TV by means of a suitable algorithm or manually based e.g. on transthoracic echocardiogram or ultrasound imaging.

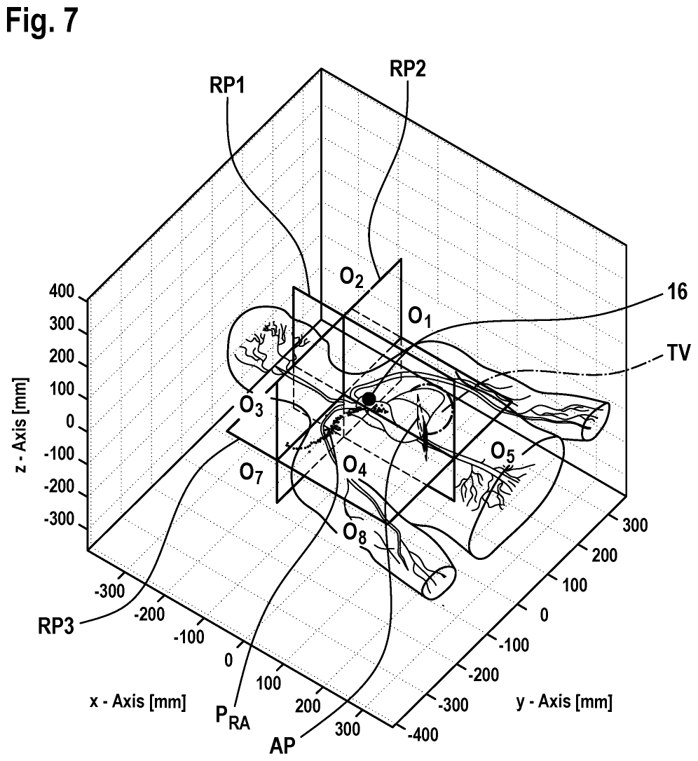

In addition to the target volume TV, the controller 18 may be further configured to establish a spatially limited auxiliary plane AP to further aid the physician during the insertion procedure of the guidewire 24 into the patient's body B. The position of the auxiliary plane AP indicates a unique structure within the patient's body B, e.g. the aortic valve, see FIG. 7. The auxiliary plane AP may be established taking into account the position of the second reference sensor 20. It is also conceivable to set the auxiliary plane AP by means of a suitable algorithm or manually based e.g. on transthoracic echocardiogram or ultrasound imaging. Of course, further auxiliary planes and/or target volumes may also be established by the controller 18, for instance in other colors denoting e.g. the aortic arch.

FIGS. 7 to 10 depict different views which may be visualized to the physician on the display device 26 showing the movement path P_{RA} of the movable sensor 14 within the patient's body for right axillary access. Of course, the movement path P_{RA} of the movable sensor 14 is based on visualized real-time data of the positions of the movable sensor 14 within the global coordinate system COS. In other words, the movement path P_{RA} is composed of a plurality of individual positions of the movable sensor 14. As shown, the physician may switch between different representations via the input device 28, e.g. with or without virtual model of the patient's body, with or without the first reference plane RP1, with or without the second reference plane RP2, with or without the third reference plane RP3, with or without the target volume TV, or with or without the auxiliary plane AP.

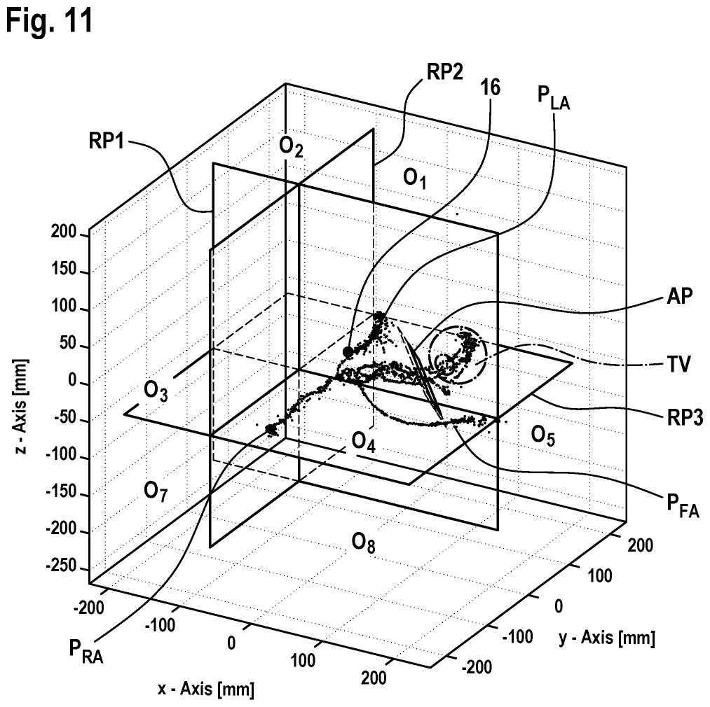

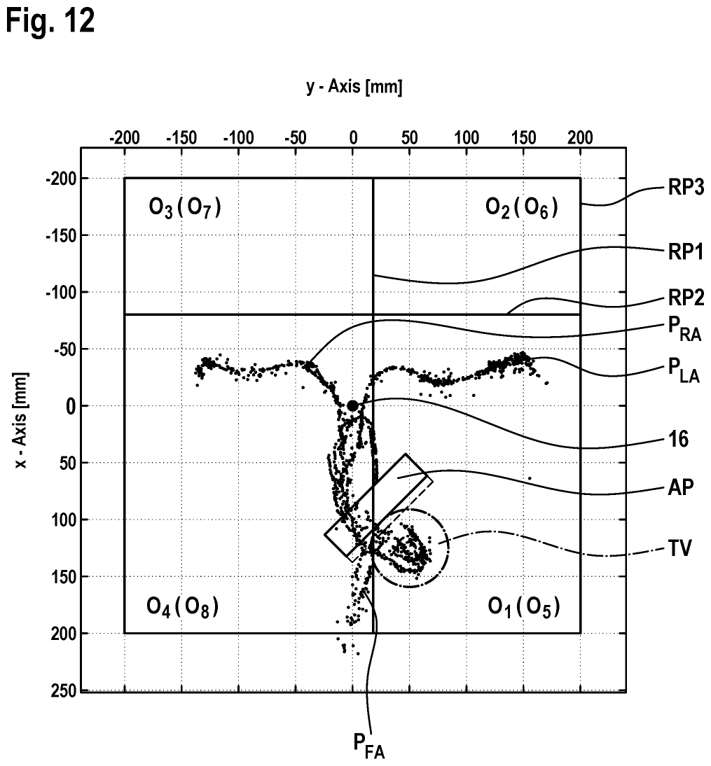

FIGS. 11 and 12 additionally show the movement path P_{LA} of the movable sensor 14 for a left axillary access and the movement path P_{FA} of the movable sensor 14 for femoral access. As mentioned, the movable sensor 14 may be attached to a guidewire 24 or also directly to an intravascular blood pump. In particular, especially during immediate care surgery it is sometimes temporally not possible to introduce first the guidewire 24 into the patient's body. In such a case, an intravascular blood pump with thereto attached movable sensor 14 may directly be introduced into the patient's body B e.g. through a femoral access. Displaying the movement path of the intravascular blood pump in real-time greatly aids the physician in correctly placing the intravascular blood pump in the patient's left ventricle.

### Exemplary Implementations

As already described, the technology described herein may be implemented in various ways. In that regard, the foregoing disclosure is intended to include, but not be limited to, the systems, methods, and combinations and subcombinations thereof that are set forth in the following exemplary implementations. Preferred embodiments are described in the following paragraphs:

A1 Tracking system (10) comprising:
an emitting device (12) configured to establish a measurement volume within at least a part of a patient's body (B), the patient's body having a median plane (MP), a transverse plane (TP), and a coronal plane (CP);
a movable sensor (14) which is movable within the measurement volume;
a first reference sensor (16) establishing a global coordinate system (COS) within the measurement volume, the global coordinate system (COS) having an origin, an x′-axis, a y′-axis and a z′-axis;
a controller (18) being configured
   to detect real-time positions of the movable sensor (14) within the global coordinate system,
   to set the origin of the global coordinate system (COS) as an anchor point in relation to the patient's body,
   to span a first reference plane (RP1) in direction of the x′-axis and the z′-axis being parallel to the median plane (MP),
   to span a second reference plane (RP2) in direction of the y′-axis and the z′-axis being parallel to the transverse plane (TP),
   to span a third reference plane (RP3) in direction of the x′-axis and the y′-axis being parallel to the coronal plane (CP), and preferably
   to translate in parallel at least one of the first reference plane (RP1), the second reference plane (RP2) and the third reference plane (RP3) based on an expected movement path of the movable sensor (14).

A2 Tracking system (10) according to paragraph A1, wherein the first reference plane (RP1), the second reference plane (RP2) and the third reference plane (RP3) establish eight octants (O₁-O₈) within the measurement volume and the controller (18) is configured to adjust the partition of the octants (O₁-O₈) based on the expected movement path of the movable sensor (14).

A3 Tracking system (10) according to paragraph A1 or A2, wherein the tracking system (10) further comprises a second reference sensor (20) and wherein the controller (18) is further configured to detect the position of the second reference sensor (20) within the global coordinate system (COS).

A4 Tracking system (10) according to any one of the preceding paragraphs A1 to A3, wherein the controller (18) is further configured
to align the x′-axis in parallel with a line of intersection (LI_{CP-MP}) of the coronal plane (CP) and the median plane (MP), and/or
to align the y′-axis in parallel with a line of intersection (LI_{CP-TP}) of the transverse plane (TP) and the coronal plane (CP), and/or
to align the z′-axis in parallel with a line of intersection (LI_{MP-TP}) of the median plane (MP) and the transverse plane (TP).

A5 Tracking system (10) according to any one of the preceding paragraphs A1 to A4, wherein the first reference sensor (16) is configured to be manually aligned with the median plane (MP) and/or the transverse plane (TP) and/or the coronal plane (CP) such that the x′-axis is parallel with a line of intersection (LI_{CP-MP}) of the coronal plane (CP) and the median plane (MP), and/or such that the y′-axis is parallel with a line of intersection (LI_{CP-TP}) of the transverse plane (TP) and the coronal plane (CP), and/or such that z′-axis is parallel with a line of intersection (LI_{MP-TP}) of the median plane (MP) and the transverse plane (TP).

A6 Tracking system (10) according to any one of the preceding paragraphs A1 to A5, further comprising a third reference sensor (22), the controller (18) being configured to set an orientation of the median plane (MP), of the transverse plane (TP), and of the coronal plane (CP) within the measurement volume based on the orientation of the third reference sensor (22).

A7 Tracking system (10) according to any one of the preceding paragraphs A1 to A6, wherein the controller (18) is configured to detect a crossing of the movable sensor (14) with at least one of the first reference plane (RP1), the second reference plane (RP2) and the third reference plane (RP3).

A8 Tracking system (10) according to any one of the preceding paragraphs A1 to A7, wherein the controller (18) is further configured to establish a target volume (TV) within the global coordinate system (COS).

A9 Tracking system (10) according to any one of the preceding paragraphs A1 to A8, wherein the controller (18) is further configured to establish a spatially limited auxiliary plane (AP), the auxiliary plane (AP) indicating a unique structure within the patient's body.

A10 Tracking system (10) according to any one of the preceding paragraphs A1 to A9, wherein the first reference sensor (16) is a six degrees of freedom sensor and/or wherein the second reference sensor (20) is a six degrees of freedom sensor.

A11 Tracking system (10) according to any one of the preceding paragraphs A1 to A8, further comprising a medical device (24), wherein the movable sensor (14) is attached to the medial device (24), and wherein the medical device (24) is preferably an intravascular blood pump or a guidewire, for example for an intravascular blood pump.

A12 Tracking system (10) according to any one of the preceding paragraphs A1 to A11, wherein the emitting device (12) is an electromagnetic field generator.

A13 Tracking system (10) according to any one of the preceding paragraphs A1 to A12, wherein the movable sensor (14) is an embedded electromagnetic sensor and/or wherein the movable sensor (14) is a five degrees of freedom sensor or a six degrees of freedom sensor.

A14 Tracking system (10) according to any one of the preceding paragraphs A1 to A13, further comprising a display device (26) configured to display the real-time positions of the movable sensor (14) and/or to display the first reference plane (RP1) and/or to display the second reference plane (RP2) and/or to display the third reference plane (RP3) and/or to display the target volume (TV) and/or to display the auxiliary plane (AP) in the global coordinate system (COS) and/or to display the octants (O₁-O₈), in particular to display the real-time positions of the movable sensor (14) in relation to the first reference plane (RP1) and/or in relation to the second reference plane (RP2) and/or in relation to the third reference plane (RP3) and/or in relation to the target volume (TV) and/or in relation to the auxiliary plane (AP) in the global coordinate system (COS) and/or in relation to the octants (O₁-O₈).

A15 Tracking system (10) according to any one of the preceding paragraphs A1 to A14, further comprising an input device (28) configured to communicate with the controller (18).

A16 Method of tracking real-time positions of a movable sensor (14) within a patient's body (B) with a tracking system (10), for example with a tracking system according to any one of the preceding paragraphs A1 to A15, the method comprising the following steps:
providing an emitting device (12) configured to establish a measurement volume within at least a part of a patient's body (B), the patient's body having a median plane (MP), a transverse plane (TP), and a coronal plane (CP),
providing a movable sensor (14) which is movable within the measurement volume,
providing a first reference sensor (16) establishing a global coordinate system (COS) within the measurement volume, the global coordinate system (COS) having an origin, an x′-axis, a y′-axis and a z′-axis,
providing a controller (18),
attaching the reference sensor (16) on the skin of the patient's body (B),
setting, with the controller (18), the origin of the global coordinate system (COS) as an anchor point in relation to the patient's body,
spanning, with the controller (18), a first reference plane (RP1) in direction of the x′-axis and the z′-axis being parallel to the median plane (MP),
spanning, with the controller (18), a second reference plane (RP2) in direction of the y′-axis and the z′-axis being parallel to the transverse plane (TP),
spanning, with the controller (18), a third reference plane (RP3) in direction of the x′-axis and the y′-axis being parallel to the coronal plane (CP),
translating, with the controller (18), in parallel at least one of the first reference plane (RP1), the second reference plane (RP2) and the third reference plane (RP3) based on an expected movement path of the movable sensor (14), and
detecting, with the controller (18), real-time positions of the movable sensor (14) within the global coordinate system (COS).

A17 Method according to paragraph A16, comprising the following steps:
establishing, with the controller (18), eight octants (O₁-O₈) within the measurement volume based on the first reference plane (RP1), the second reference plane (RP2) and the third reference plane (RP3),
adjusting, with the controller (18) the partition of the octants (O₁-O₈) based on the expected movement path of the movable sensor (14).

A18 Method according to paragraph A16 or A17, comprising the following steps:
providing a second reference sensor (20), and
detecting, with the controller (18), the position of the second reference sensor (20) within the global coordinate system (COS).

A19 Method according to any one of the preceding paragraphs A16 to A18, comprising the following steps:
aligning the x′-axis in parallel with a line of intersection (LI_{CP-MP}) of the coronal plane (CP) and the median plane (MP), and/or
aligning the y′-axis in parallel with a line of intersection (LI_{CP-TP}) of the transverse plane (TP) and the coronal plane (CP), and/or
aligning the z′-axis in parallel with a line of intersection (LI_{MP-TP}) of the median plane (MP) and the transverse plane (TP).

A20 Method according to any one of the preceding paragraphs A16 to A19, comprising the following steps: manually aligning the first reference sensor (16) with the median plane (MP) and/or the transverse plane (TP) and/or the coronal plane (CP) such that the x′-axis is parallel with a line of intersection (LI_{CP-MP}) of the coronal plane (CP) and the median plane (MP), and/or such that the y′-axis is parallel with a line of intersection (LI_{CP-TP}) of the transverse plane (TP) and the coronal plane (CP), and/or such that z′-axis is parallel with a line of intersection (LI_{MP-TP}) of the median plane (MP) and the transverse plane (TP).

A21 Method according to any one of the preceding paragraphs A16 to A20, comprising the following steps:
providing a third reference sensor (22), and
setting, with the controller (18), an orientation of the median plane (MP), an orientation of the transverse plane (TP), and/or an orientation of the coronal plane (CP) within the measurement volume based on the orientation of the third reference sensor (22).

A22 Method according to any one of the preceding paragraphs A16 to A21, comprising the steps:
detecting, with the controller (18), a crossing of the moveable sensor (14) with at least one of the first reference plane (RP1), the second reference plane (RP2) and the third reference plane (RP3).

A23 Method according to any one of the preceding paragraphs A16 to A22, comprising the steps
establishing, with the controller (18), a target volume (TV) within the global coordinate system (COS).

A24 Method according to any one of the preceding paragraphs A16 to A23, comprising the following steps:
establishing, with the controller (18), a spatially limited auxiliary plane (AP), the auxiliary plane (AP) indicating a unique structure within the patient's body (B).

A25 Method according to any one of the preceding paragraphs A16 to A24, comprising the following steps:
displaying, on a display device (26), the real-time positions of the movable sensor (14) and/or displaying, on a display device (26), the first reference plane (RP1) and/or displaying, on a display device (26), the second reference plane (RP2) and/or displaying, on a display device (26), the third reference plane (RP3) and/or displaying, on a display device (26), the target volume (TV) and/or displaying, on a display device (26), the auxiliary plane (AP) in the global coordinate system (COS) and/or displaying, on a display device (26), the octants (O₁-O₈).

A26 Method according to paragraph A25, wherein displaying on the display device (26) includes displaying the real-time positions of the movable sensor (14) in relation to the first reference plane (RP1) and/or in relation to the second reference plane (RP2) and/or in relation to the third reference plane (RP3) and/or in relation to the target volume (TV) and/or in relation to the auxiliary plane (AP) in the global coordinate system (COS) and/or in relation to the octants (O₁-O₈).

A27 Method according to any one of the preceding paragraphs A16 to A26, wherein the movable sensor (14) is introduced into the patient's body (B) via the femoral artery or via the axillary artery.

### LIST OF REFERENCE SIGNS

10 tracking system
12 emitting device/electromagnetic field generator
14 movable sensor
16 first reference sensor
18 controller
20 second reference sensor
22 third reference sensor
24 medical device
26 display device
28 input device
AP auxiliary plane
COS global coordinate system
CP coronal plane
LI_{CP-MP} line of intersection between coronal plane and median plane
LI_{CP-TP} line of intersection between coronal plane and transverse plane
LI_{MP-TP} line of intersection between median plane and transverse plane
MP median plane
TP transverse plane
O₁-O₈ octants
P_{FA} movement path of movable sensor for femoral access
P_{LA} movement path of movable sensor for left axillary access
P_{RA} movement path of movable sensor for right axillary access
RP1 first reference plane
RP2 second reference plane
RP3 third reference plane
TV target volume

1. A tracking system comprising:
an emitting device configured to establish a measurement volume within at least a part of a patient's body, the patient's body having a median plane, a transverse plane, and a coronal plane;
a movable sensor which is movable within the measurement volume;
a first reference sensor establishing a global coordinate system within the measurement volume, the global coordinate system having an origin, an x′-axis, a y′-axis and a z′-axis; and
a controller configured to:
detect real-time positions of the movable sensor within the global coordinate system;
set the origin of the global coordinate system as an anchor point in relation to the patient's body;
span a first reference plane in direction of the x′-axis and the z′-axis being parallel to the median plane;
span a second reference plane in direction of the y′-axis and the z′-axis being parallel to the transverse plane;
span a third reference plane in direction of the x′-axis and the y′-axis being parallel to the coronal plane; and
translate in parallel at least one of the first reference plane, the second reference plane or the third reference plane based on an expected movement path of the movable sensor.

2. A tracking system according to claim 1, wherein the first reference plane, the second reference plane and the third reference plane establish eight octants within the measurement volume, and wherein the controller is configured to adjust a partition of the eight octants based on the expected movement path of the movable sensor.

3. A tracking system according to claim 1, further comprising a second reference sensor, and wherein the controller is further configured to detect a position and/or an orientation of the second reference sensor within the global coordinate system.

4. A tracking system according to claim 1, wherein the controller is further configured to:
align the x′-axis in parallel with a line of intersection of the coronal plane and the median plane; and/or
align the y′-axis in parallel with a line of intersection of the transverse plane and the coronal plane; and/or
align the z′-axis in parallel with a line of intersection of the median plane and the transverse plane.

5. A tracking system according to claim 1, wherein the first reference sensor is configured to be manually aligned with the median plane and/or the transverse plane and/or the coronal plane such that the x′-axis is parallel with a line of intersection of the coronal plane and the median plane, and/or such that the y′-axis is parallel with a line of intersection of the transverse plane and the coronal plane, and/or such that the z′-axis is parallel with a line of intersection of the median plane and the transverse plane.

6. A tracking system according to claim 1, further comprising a third reference sensor, the controller being configured to set an orientation of the median plane, an orientation of the transverse plane, and/or an orientation of the coronal plane within the measurement volume based on the orientation of the third reference sensor.

7. A tracking system according to claim 1, wherein the controller is further configured to detect a crossing of the movable sensor with at least one of the first reference plane, the second reference plane or the third reference plane.

8. A tracking system according to claim 1, wherein the controller is further configured to establish a target volume within the global coordinate system.

9. A tracking system according to claim 1, wherein the controller is further configured to establish a spatially limited auxiliary plane, the auxiliary plane indicating a unique structure within the patient's body.

10. A tracking system according to claim 3, wherein the first reference sensor is a six degrees of freedom sensor and/or the second reference sensor is a six degrees of freedom sensor.

11. A tracking system according to claim 1, further comprising a medical device, wherein the movable sensor is attached to the medical device, and wherein the medical device comprises an intravascular blood pump or a guidewire for an intravascular blood pump.

12. A tracking system according to claim 1, wherein the emitting device is an electromagnetic field generator.

13. A tracking system according to claim 1, wherein the movable sensor is an embedded electromagnetic sensor and/or wherein the movable sensor is a five degrees of freedom sensor or a six degrees of freedom sensor.

14. A tracking system according to claim 1, further comprising a display device configured to display one or more of the real-time positions of the movable sensor, the first reference plane, the second reference plane, the third reference plane, a target volume, an auxiliary plane in the global coordinate system, octants within the measurement volume, or the real-time positions of the movable sensor in relation to one or more of the first reference plane, the second reference plane, the third reference plane, the target volume, the auxiliary plane in the global coordinate system or the octants.

15. A tracking system according to claim 1, further comprising an input device configured to communicate with the controller.
